(19) United States
(12) Patent Application Publication
(10) Pub. No.: US 2025/0353637 A1
(43) Pub. Date: Nov. 20, 2025

(54) Tray Assembly

(71) Applicant: HR Pharmaceuticals, Inc., York, PA (US)

(72) Inventors: Mathew Hanna, Lancaster, PA (US); Colby Wiesman, Wrightsville, PA (US); Chris Wiesman, York, PA (US)

(73) Assignee: HR Pharmaceuticals, Inc., York, PA (US)

(21) Appl. No.: 18/664,820

(22) Filed: May 15, 2024

(51) Int. Cl. B65D 1/36

(52) U.S. Cl. CPC B65D 1/36

(57) ABSTRACT

A tray includes a first layer having a plurality of compartment. The tray includes a second layer non-removably coupled to the first layer. A container is positioned within the interior of the second layer. A cover attachable to an opening of the second layer. The cover having a passageway extending through the cover and into the interior of the second layer.

### FIELD OF THE INVENTION

The present invention relates to a tray assembly and, more particularly, to a tray assembly to aid in the utilization of a foley catheter.

### BACKGROUND

In general, urinary drainage systems are conventionally used in hospitals and health care facilities when it is necessary to facilitate, control, monitor urination of patients, and when it is necessary to collect urine from a patient. These urinary drainage systems permit the patient to remain in bed, without having to use a bedpan or be moved to use a bathroom. Urinary drainage systems may include a Foley catheter, a collection container/bag (e.g., a bag made of a polymeric material or PVC film) and tubing connecting the Foley catheter to the collection bag.

In operation, the patient is first catheterized, and the Foley catheter is connected to the drainage bag through a length of tubing. The urine drains through the Foley catheter, the tubing, and then finally into the collection bag.

As the foregoing illustrates, the invention provides the tray assembly to permit useful transition from preparing the patient to executing a successful urine collection.

### SUMMARY

A tray includes a first layer having a plurality of compartment. The tray includes a second layer non-removably coupled to the first layer. A container is positioned within the interior of the second layer. A cover attachable to an opening of the second layer. The cover having a passageway extending through the cover and into the interior of the second layer.

### BRIEF DESCRIPTION OF DRAWINGS

In the following, the present invention is described in more detail with references to the drawings in which:

FIG. 1 illustrates a front, top, left side perspective view of the invention;

FIG. 2 illustrates a top view of the invention of FIG. 1;

FIG. 3 illustrates another embodiment of the invention;

FIG. 4 illustrates another embodiment of the invention;

FIG. 5 illustrates a top view of the invention of FIG. 4;

FIG. 6 illustrates a right side view of the invention;

FIG. 7 illustrates a left side view of the invention;

FIG. 8 illustrates a rear view of the invention;

FIG. 9 illustrates a front view of the invention;

FIG. 10 illustrates a top sectional view of a portion of the invention;

FIG. 11 illustrates a top view with a plurality of components positioned within; and

FIG. 12 illustrates a front view with the plurality of components positioned within.

### DETAILED DESCRIPTION OF THE EMBODIMENTS

The present disclosure includes a tray assembly 1 according to the invention. In the exemplary embodiment, the tray assembly 1 includes a first layer 2 and a second layer 4.

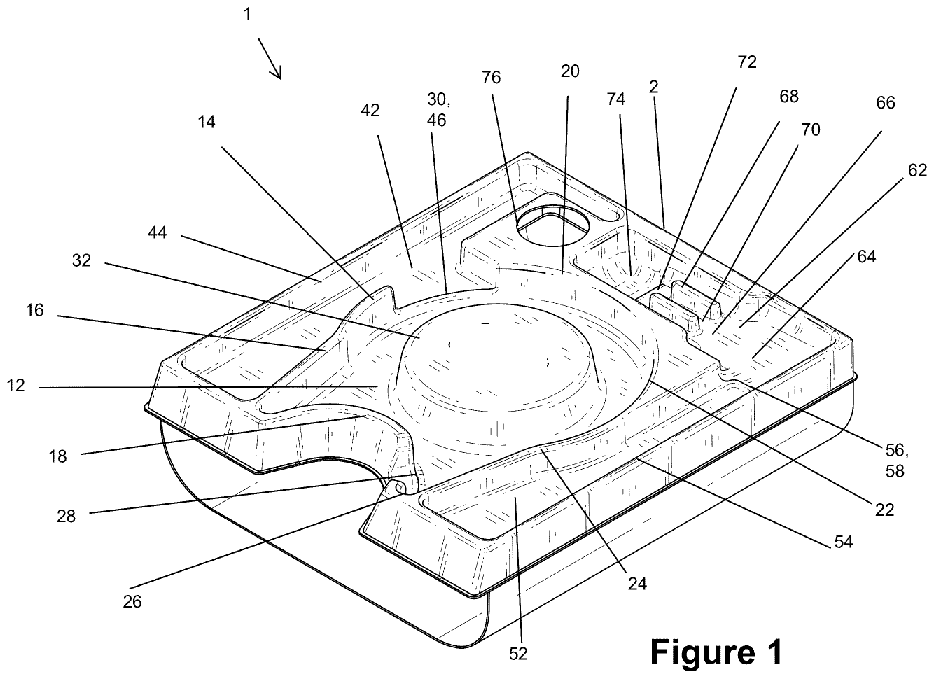

As shown in FIG. 1, the first layer 2 is a frame 2. The frame 2 includes a first compartment 10, a second compartment 40, a third compartment 50 and a fourth compartment 60.

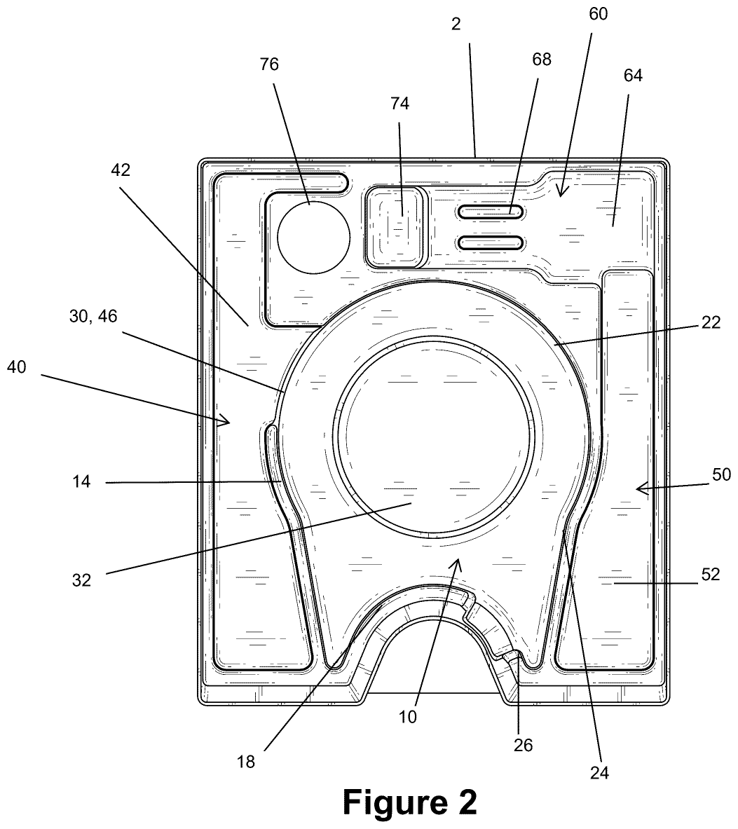

In the exemplary embodiment and shown in FIGS. 1-2, the first compartment 10 includes a first base member 12. The first base member 12 is composed partially of a bottom portion of the frame 2. The first base member 12 is planar.

The first compartment 10 further includes a first compartment first barrier wall 14. The first compartment first barrier wall 14 extends above the first base member 12. The first compartment first barrier wall 14 further extends around a portion of the first base member 12. The first compartment first barrier wall 14 includes an almost entirely straight portion 16. The first compartment first barrier wall 14 further includes a curve portion 18 coupling to the straight portion 16. One skilled in the art would understand the applicant's design is not the exclusive embodiment.

The first compartment 10 further includes a first compartment second barrier wall 20. The first compartment second barrier wall 20 extends above the first base member 12. The first compartment second barrier wall 20 further extends along a portion of the first base member 12. The first compartment second barrier wall 20 includes a curvature portion 22 composing most of the first compartment second barrier wall 20. The first compartment second barrier wall 20 further includes an almost straight portion 24 coupling to the curvature portion 22. The first compartment second barrier wall 20 further includes a hook shaped portion 26 coupling to the straight portion 24.

The first compartment 10 further includes a first opening 28. The first opening 28 is formulated by the first compartment first barrier wall 14 and the first compartment second barrier wall 20. The first opening 28 extends through the perimeter of the frame 2.

The first compartment 10 further includes a second opening 30. The second opening 30 is formulated by the first compartment first barrier wall 14 and the first compartment second barrier wall 20. The second opening 30 extends through and into the second compartment 40.

The first compartment 10 further includes a central protruding member 32. The central protruding member 32 is circular and extends away from the frame 2.

In the exemplary embodiment, the second compartment 40 includes a second base member 42. The second base member 42 is composed partially of a bottom portion of the frame 2.

The second compartment 40 further includes a second compartment barrier wall 44 which extends almost an entire perimeter of the second base member 42. A portion of the first compartment first barrier wall 14 is shared with a portion of the second compartment barrier wall 44.

The second compartment 40 further includes a lip 46. The lip 46 aligns with the second opening 30 and is a semi-circular groove which tapers towards the first compartment 10 allowing an avenue between the first compartment 10 and the second compartment 40.

In the exemplary embodiment, the third compartment 50 includes a third base member 52. The third base member 52 is composed partially of the bottom portion of the frame 2.

The third compartment 50 further includes a third compartment barrier wall 54. The third compartment barrier wall 54 extends along almost an entire outer portion of the third base member 52.

The third compartment 50 further includes a ledge 56. The ledge 56 divides the third compartment 50 from the fourth compartment and permits a channel 58 between the third compartment 50 and the fourth compartment 60. The ledge 56 tapers towards the fourth compartment.

In the exemplary embodiment, the fourth compartment 60 includes a fourth base member 62. The fourth base member 62 is composed partially of the bottom portion of the frame 2. The fourth base member 62 as shown, has an overall declination. The fourth base member 62 further includes an upper portion 64, a central portion 66 and a lower portion 74.

The upper portion 64 of the fourth base member 62 includes a height less than the height of the first base member 12, the second base member 42 and the third base member 52.

The central portion 66 includes a pair of protruding walls 68. The pair of protruding walls 68 are elongated members extending in a parallel direction to the fourth base member 62, specifically, each of the protruding walls is cylindrical. One skilled in the art would understand the applicant's design is not the exclusive embodiment. The pair of protruding walls 68 define a plurality of slots 70 as shown. One skilled in the art would understand the applicant's design is not the exclusive embodiment.

The central portion 66 further includes a ledge 72, the ledge 72 divides the central portion 66 and the lower portion 74. The ledge 72 tapers towards the lower portion 74.

The lower portion 74 of the fourth base member 62 is a rectangular concavity extending downwards towards the second layer 4.

The frame 2 further includes a passageway 76. The passageway 76 is circular and positioned between the first compartment second barrier wall 20 and the second compartment barrier wall 44. The passageway 76 extends through the first layer 2 and into the second layer 4.

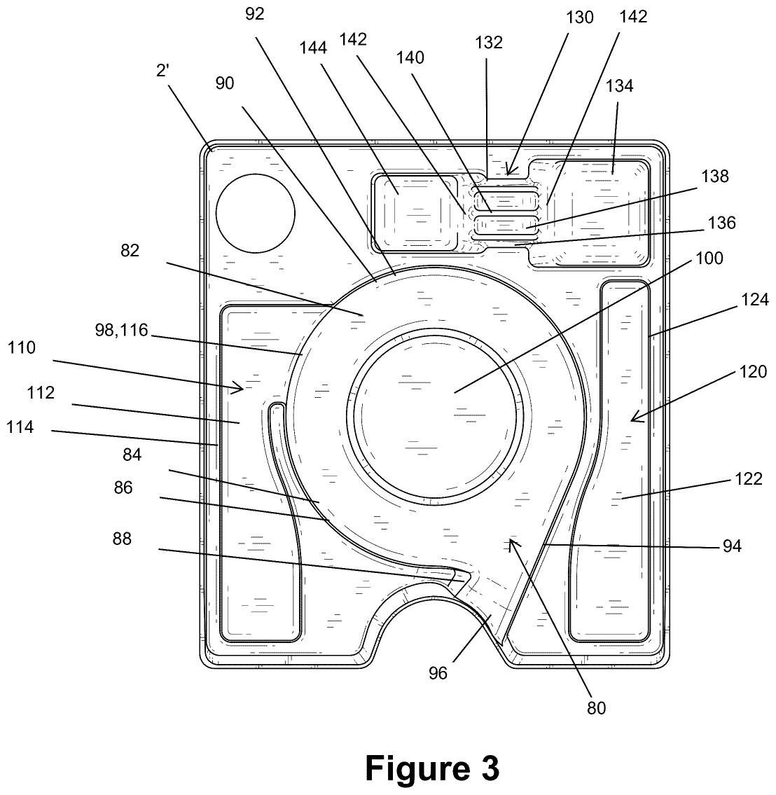

In another exemplary embodiment of first layer as shown in FIG. 3, the first layer is a frame 2′. The frame 2′ includes a first compartment 80, a second compartment 110, a third compartment 120 and a fourth compartment 130.

The first compartment 80 includes a first base member 82. The first base member 82 is composed partially of a bottom portion of the frame 2′. The first base member 82 is planar.

The first compartment 80 further includes a first compartment first barrier wall 84. The first compartment first barrier wall 84 extends above the first base member 82. The first compartment first barrier wall 84 further extends around a portion of the first base member 82. The first compartment first barrier wall 84 is composed of a curvature portion 86. The curvature portion 86 is almost the entire length of the first compartment first barrier wall 84. The first compartment first barrier wall 84 further includes a straight portion 88. The curvature portion 86 couples into the straight portion 88 forming the first compartment first barrier wall 84.

The first compartment 80 further incudes a first compartment second barrier wall 90. The first compartment second barrier wall 90 extends above the first base member 82. The first compartment second barrier wall 90 further extends along a portion of the first base member 82 in an opposite direction from the first compartment first barrier wall 84. The first compartment second barrier wall 90 includes a curvature portion 92 which formulates at least half of the first compartment second barrier wall 90. The first compartment second barrier wall 90 further includes a straight portion 94 coupling to the curvature portion 92 to form the first compartment second barrier wall 90. The first compartment second barrier wall 90 extends a distance further than the first compartment first barrier wall 84.

The first compartment 80 further includes a first opening 96. The first opening 96 is formulated by a break between the first compartment first barrier wall 84 and the first compartment second barrier wall 90. The first opening 96 extends through the perimeter of the frame 2′.

The first compartment 80 further includes a second opening 98. Similarly, the second opening 98 is formulated by a break between the first compartment first barrier wall 84 and the first compartment second barrier wall 90. The second opening 98 extends through and into the second compartment 110.

The first compartment 80 further includes a central protruding member 100. The central protruding member 100 is circular. The central protruding member 100 is equal in height to the first compartment first barrier wall 84 and the first compartment second barrier wall 90.

The second compartment 110 includes a second base member 112. The second base member 112 is composed partially of a bottom portion of the frame 2′.

The second compartment 110 further includes a second compartment barrier wall 114. The second compartment barrier wall 114 extends above the second base member 112. The second compartment barrier wall 114 extends along an outer portion of the second base member 112. A portion of the first compartment first barrier wall 84 is shared with a portion of the second compartment barrier wall 114.

The second compartment 110 further includes a lip 116. The lip 116 is a semi-circular groove extending through both the first compartment 80 and the second compartment 110.

The third compartment 120 includes a third base member 122. The third base member 122 is composed partially of the bottom portion of the frame 2′.

The third compartment 120 further includes a third compartment barrier wall 124. The third compartment barrier wall 124 extends the entire outer portion of the third base member 122.

The fourth compartment 130 includes a fourth base member 132. The fourth base member 132 is composed partially of the bottom portion of the frame 2′. The fourth base member 132 further includes an upper portion 134, a central portion 136 and a lower portion 144.

The upper portion 134 of the fourth base member 132 is a rectangular concavity. One skilled in the art would understand the applicant's design is not the exclusive embodiment.

The central portion 136 is raised above the upper portion 134. The central portion 136 includes a pair of protruding walls 138. The pair of protruding walls 138 are elongated members extending in a parallel direction to the fourth base member 132.

The pair of protruding walls 138 define a plurality of tapering valleys 140.

The central portion 136 further includes a pair of ledges 142, dividing the upper portion 134, central portion 136 and the lower portion 144. One of the pair of ledges 142 tapers towards the upper portion 134 and the second of the pair of ledges 142 tapers towards the lower portion 144.

The lower portion 144 of the fourth base member 132 is a rectangular concavity with a perimeter less than a perimeter of the upper portion 134.

The frame 2′ further includes a passageway 150. The passageway 150 is circular and positioned between the first compartment second barrier wall 90 and the second compartment barrier wall 114. The passageway 150 extends through the first layer 2 and into the second layer 4.

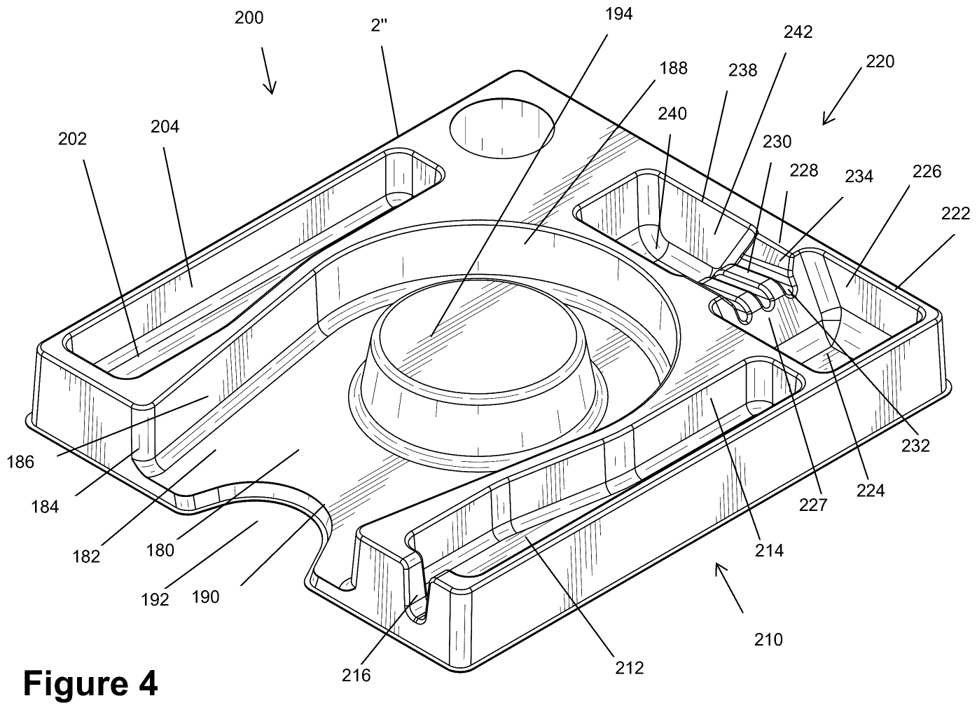

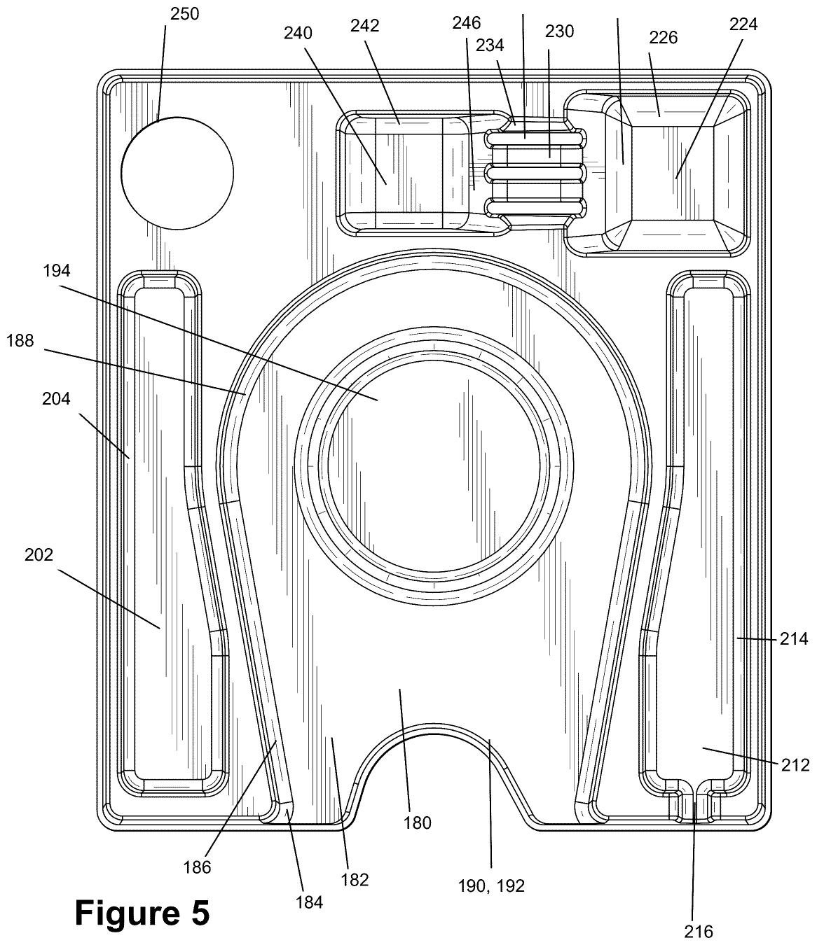

In another exemplary embodiment and shown in FIGS. 4-5, the first compartment 180 includes a first base member 182. The first base member 182 is composed partially of a bottom portion of the frame 2″. The first base member 182 is planar.

The first compartment 180 further includes a first compartment first barrier wall 184. The first compartment first barrier wall 184 extends above the first base member 182. The first compartment first barrier wall 184 further extends around a portion of the first base member 182. The first compartment first barrier wall 184 includes an almost entirely straight portion 186. The first compartment first barrier wall 184 further includes a curve portion 188 coupling to the straight portion 186. One skilled in the art would understand the applicant's design is not the exclusive embodiment.

The first compartment 180 further includes an opening 190. The opening 190 is positioned between the straight portions 186 and also extends through the perimeter of the frame 2″. The first compartment 180 further includes a passageway 192 extending through the first base member 182.

The first compartment 180 further includes a central protruding member 194. The central protruding member 194 is circular and extends away from the frame 2″.

In the exemplary embodiment, the second compartment 200 includes a second base member 202. The second base member 202 is composed partially of a bottom portion of the frame 2″.

The second compartment 200 further includes a second compartment barrier wall 204 which extends the entire perimeter of the second base member 202. The second compartment barrier wall 204 permits tapering. One skilled in the art would understand the applicant's design is not the exclusive embodiment.

In the exemplary embodiment, the third compartment 210 includes a third base member 212. The third base member 212 is composed partially of the bottom portion of the frame 2″.

The third compartment 210 further includes a third compartment barrier wall 214. The third compartment barrier wall 214 extends along almost an entire outer portion of the third base member 212. The third compartment barrier wall 214 permits tapering. The third compartment 210 further includes a u-shape passageway positioned between the third compartment barrier wall 214 and also extends through the frame 2.

In the exemplary embodiment, the fourth compartment 220 includes an upper portion 222, a central portion 228 and a lower portion 238.

The upper portion 222 includes a base member 224, the base member 224 is a rectangular concavity. The upper portion 222 further includes a plurality of side walls 226. Specifically, the at least three side walls 226 extend upwards from the base member 224. The upper portion 222 further includes a sloped side wall 227 extending away from the base member 224.

The central portion 228 includes a pair of protruding walls 230. The pair of protruding walls 230 define a plurality of slots 232 as shown in FIGS. 4-5. The plurality of slots 232 angling downwards and away from the upper portion 222. One skilled in the art would understand the applicant's design is not the exclusive embodiment.

The central portion 228 includes a pair of side walls 234 extending away from at least two slots 232.

The lower portion 238 includes a base member 240, the base member 240 is a rectangular concavity. The lower portion 238 includes a plurality of side walls 242. Specifically, the at least three side walls 242 extend upwards from the base member 240. The lower portion further includes a sloped side wall 246 extending away from the base member 240.

The frame 2″ further includes a passageway 250. The passageway is circular and positioned at a corner of the frame 2″, specifically the passageway 250 is adjacent and between the second compartment 200 and fourth compartment 220.

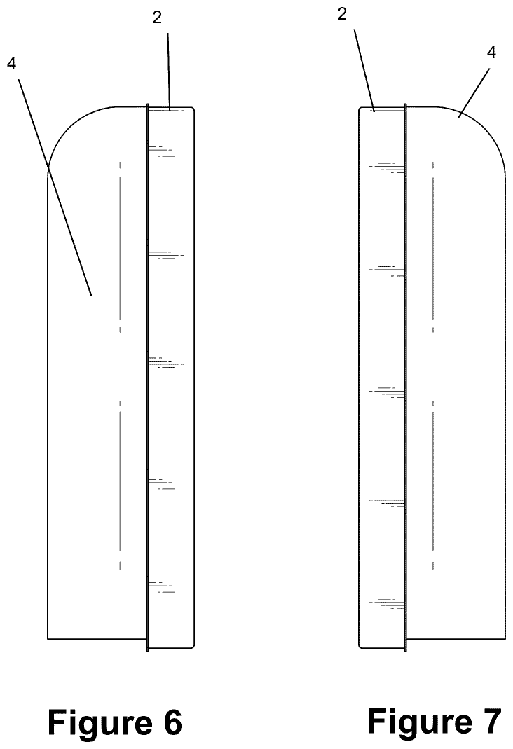

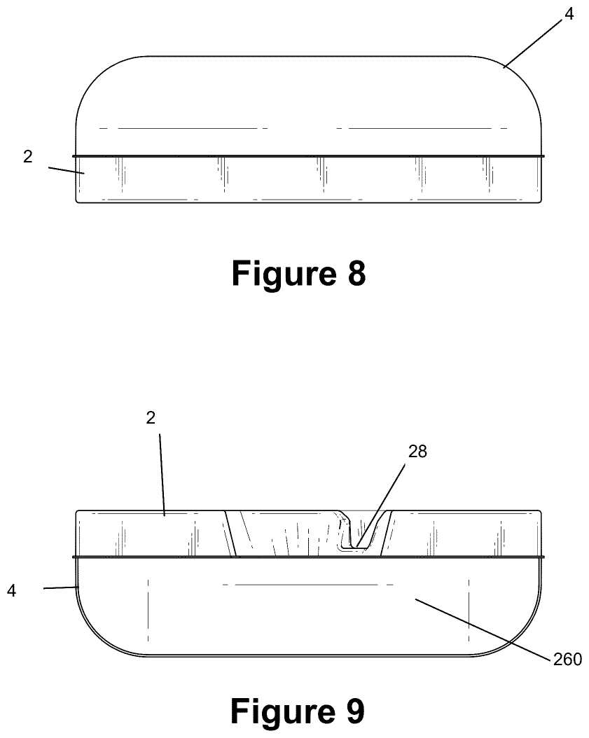

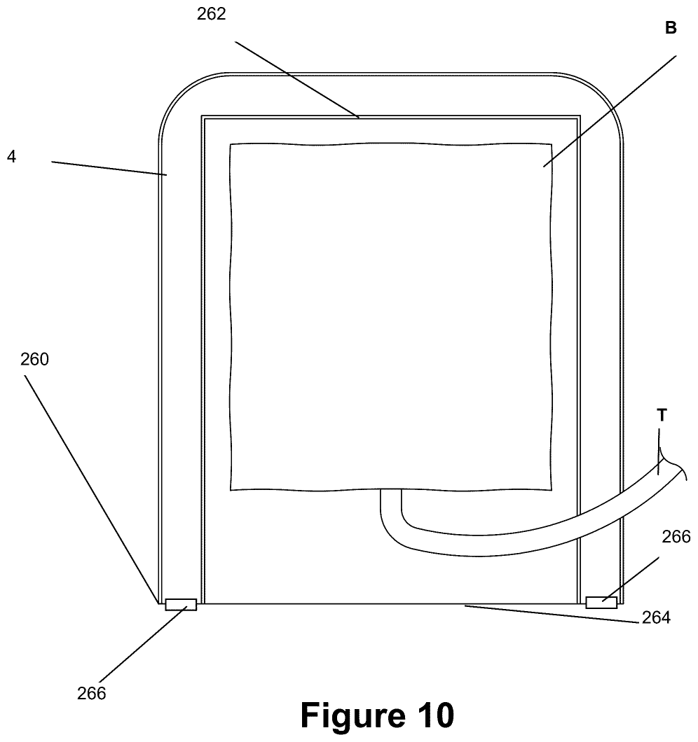

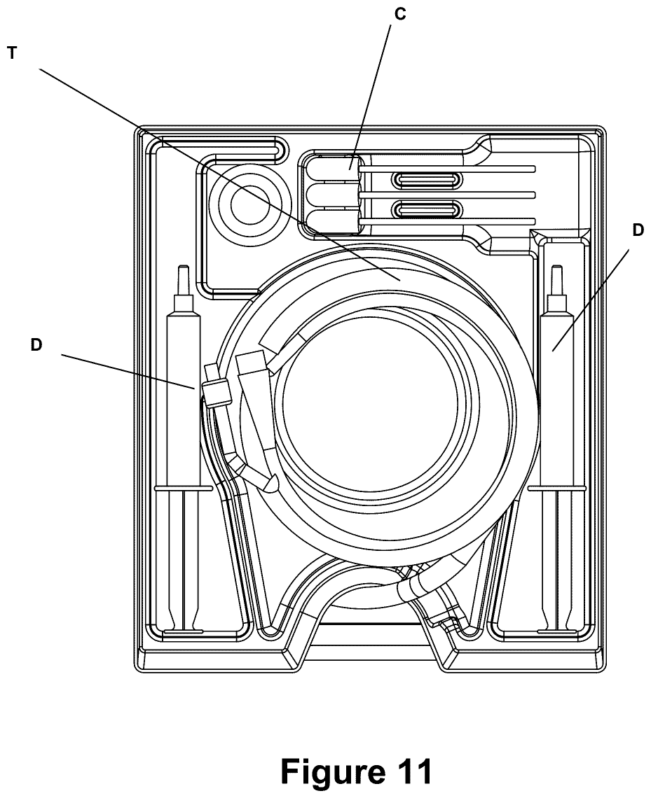

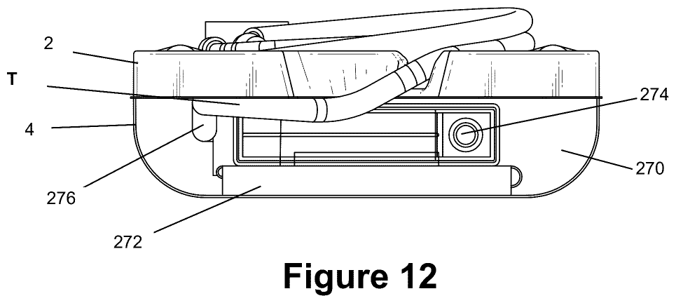

In the exemplary embodiment as shown in FIGS. 6-12, the second layer 4 is an elongated curvature member. A perimeter of the second layer 4 is positioned and coupled against the perimeter of the first layer 2, 2′,2″. The first layer 2, 2′,2″ may be coupled to the second layer 4. But for example, purposes, the first layer 2 will be utilized. The second layer 4 further provides a receiver 260. The receiver 260 is defined by an inner portion of the second layer 4. The first layer 2 slightly extends over the second layer 4, specifically in the direction of the receiver 260.

As shown in FIG. 9, the receiver 260 includes a container 262. The container 262 is a rectangular structure positioned within the second layer 4. The container 262 further includes a material opening 264 parallel to an opening of the receiver 260. In the exemplary embodiment, a bag B is positioned within the container 262. One skilled in the art would understand the applicant's design is not the exclusive embodiment.

In the exemplary embodiment, the second layer 4 further includes a latch 266. The latch 266 is positioned around the receiver 260. One skilled in the art would understand the applicant's design of the latch 266 is not the exclusive embodiment. In another exemplary embodiment, the second layer 4 does not include a latch.

As shown in FIGS. 6-12, the second layer 4 further includes a cover 270. The cover 270 is shaped to mirror the opening of the receiver 260 to allow complete coverage. The cover 270 further includes a handle 272 positioned at a lower portion of the cover 270. The handle 272 is an elongated sheet, however, one skilled in the art would understand the applicant's design is not the exclusive embodiment.

In the exemplary embodiment, the cover 270 further includes a release button 274 positioned above the handle 272 in order to release the latch 266. In another exemplary embodiment, the cover 270 does not include a release button 274. One skilled in the art would understand the applicant's design is not the exclusive embodiment.

In the exemplary embodiment, the cover 270 further includes a passageway 276 extending through the cover 270 allowing for tubing to enter the second layer 4.

As assembled in the exemplary embodiments presented, the first layer 2, 2′, or 2″ is positioned above the second layer 4. The first layer 2 is non-removable from the second layer 4. The tubing T is coiled into the first compartment 10 and around the central protruding member 32. The tubing T is then threaded through the first opening 28 and into the passageway 176 of the cover 170, allowing the tubing T to enter the second layer 4. The tubing T couples to the bag B positioned within the container 262. A fluid device D is positioned within the second compartment 40 and the third compartment 50 of the frame 2. A plurality of cotton swabs C are positioned between the pair of protruding walls 68 and into the slots 70. The plurality of cotton swabs C extend towards the lower portion 74 of the fourth base member 62. One skilled in the art would understand the applicant's design is not the exclusive embodiment.

As assembled in another exemplary embodiments presented, the frame 2′ ie. first layer 2 is positioned above the second layer 4. The first layer 2 is non-removable from the second layer 4. The tubing T is coiled into the first compartment 80 and around the central protruding member 100. The tubing T is then threaded through the first opening 96 and into the passageway 176 of the cover 170, allowing the tubing T to enter the second layer 4. The tubing T couples to the bag B positioned within the container 262. A fluid device D is positioned within the second compartment 110 and the third compartment 120 of the frame 2′. A plurality of cotton swabs C are positioned between the pair of protruding walls 138 and into the plurality of tapering valleys 140. The plurality of cotton swabs C extend towards the lower portion 144 of the fourth base member 132. One skilled in the art would understand the applicant's design is not the exclusive embodiment.

As assembled in another exemplary embodiment presented, the frame 2″ ie. first layer 2 is positioned above the second layer 4. The first layer 2 is non-removable from the second layer 4. The tubing T is coiled into the first compartment 180 and around the central protruding member 194. The tubing T is then threaded through the passageway 192 and into the passageway 176 of the cover 170, allowing the tubing T to enter the second layer 4. The tubing T couples to the bag B positioned within the container 262. A fluid device D is positioned within the second compartment 200 and the third compartment 210 of the frame 2″. A plurality of cotton swabs C are positioned between the pair of protruding walls 230 and into the plurality of slots 232. The plurality of cotton swabs C extend towards the lower portion 238 and into the base member 240. One skilled in the art would understand the applicant's design is not the exclusive embodiment.

It should be noted that various changes and modifications to the embodiments described herein will be apparent to those skilled in the art. Such changes and modifications may be made without departing from the spirit and scope of the present invention and without diminishing its intended advantages. For example, various embodiments of the systems and methods may be provided based on various combinations of the features and functions from the subject matter provided herein.

What is claimed is:

1. A tray comprising:
a first layer having a plurality of compartments;
a second layer, non-removably coupled to the first layer; a container is positioned within an interior of the second layer;
a cover attachable to an opening of the second layer; the cover having a passageway extending through the cover and into the interior of the second layer.

2. The tray of claim 1, wherein the first layer is a frame.

3. The tray of claim 2, wherein the plurality of compartments includes a first compartment, a second compartment, a third compartment and a fourth compartment.

4. The tray of claim 3, wherein the first compartment is positioned between the second compartment and the third compartment.

5. The tray of claim 4, wherein the first compartment includes a first compartment first barrier wall and a first compartment second barrier wall.

6. The tray of claim 5, wherein the first compartment first barrier wall and the first compartment second barrier wall form a first opening extending through a perimeter of the frame.

7. The tray of claim 6, wherein the first compartment first barrier wall and the first compartment second barrier wall form a second opening extending through both the first compartment and the second compartment.

8. The tray of claim 7, wherein the first compartment further includes a central protruding member that extends away from the frame.

9. The tray of claim 8, wherein the second compartment includes a second compartment barrier wall extending almost an entire perimeter of the second compartment.

10. The tray of claim 9, wherein the second compartment further includes a lip tapering towards the first compartment, the lip aligns with the second opening.

11. The tray of claim 10, wherein the third compartment includes a third compartment barrier wall which extends almost an entire perimeter of the third compartment.

12. The tray of claim 11, wherein the third compartment further includes a ledge, the ledge divides the third compartment from the fourth compartment and permits a channel between the third compartment and the fourth compartment.

13. The tray of claim 12, wherein the fourth compartment includes an upper portion, a central portion and a lower portion.

14. The tray of claim 13, wherein the portions of the fourth compartment are on a declining plane.

15. The tray of claim 14, wherein the central portion includes a pair of protruding walls.

16. The tray of claim 15, wherein the central portion further includes a ledge, the ledge divides the central portion and the lower portion.

17. The tray of claim 16, wherein the lower portion is a rectangular concavity.

18. The tray of claim 17, wherein the frame further includes a passageway 76 extending through the frame and into the second layer.

19. The tray of claim 18, wherein the cover further includes a handle.
